(19) United States
(12) Patent Application Publication
(10) Pub. No.: US 2022/0040196 A1
(43) Pub. Date: Feb. 10, 2022

(54) Pharmaceutical Composition of Chlordiazepoxide and Clidinium Combination

(71) Applicant: Jubilant Pharma Holdings Inc., Yardley, PA (US)

(72) Inventors: Indranil Nandi, Yardley, PA (US); Anil Jain, Noida (IN); Saurabh Srivastava, Noida (IN); Munish Dhiman, Noida (IN)

(73) Assignee: Jubilant Pharma Holdings Inc., Yardley, PA (US)

(21) Appl. No.: 17/396,593

(22) Filed: Aug. 6, 2021

(30) Foreign Application Priority Data
Aug. 10, 2020 (IN) 202011034293

(51) Int. Cl. A61K 31/5513; A61K 31/46; A61K 9/48; A61K 9/00; A61J 1/03; A61J 3/07; A61J 3/00

(52) U.S. Cl. CPC A61K 31/5513; A61K 31/46; A61K 9/4833; A61J 3/00; A61J 1/03; A61J 1/035; A61J 3/07; A61K 9/009

(57) ABSTRACT

The present invention relates to a stable pharmaceutical composition comprising a combination of chlordiazepoxide hydrochloride and clidinium bromide and one or more pharmaceutically acceptable excipients, wherein the composition is in the form of solid oral dosage forms like capsule and sachet. The technical challenges like undesirable impurities in dosage form were successfully controlled by using: a) low moisture excipients; and b) formulation development under controlled temperature and controlled humidity conditions. The formulations as per the present invention are stable and exhibit desired pharmaceutical technical attributes like assay and dissolution. The prepared formulations are useful for the treatment of gastrointestinal disorders and various other therapeutic indications as described herein.

### FIELD OF THE INVENTION

The present invention relates to a stable pharmaceutical composition comprising a combination of chlordiazepoxide and clidinium or their pharmaceutically acceptable salts thereof and at least one low moisture excipient. The invention also provides a process for manufacturing such compositions and further relates to the use of said composition in the treatment of gastrointestinal disorders and various other therapeutic indications as described herein.

### BACKGROUND OF THE INVENTION

Chlordiazepoxide hydrochloride is a versatile, therapeutic agent of proven value for the relief of anxiety and tension. Chlordiazepoxide hydrochloride is a BCS class II drug and is chemically described as 7-chloro-2-methylamino-5-phenyl-3H-1, 4-benzodiazepine 4-oxide hydrochloride. The structural formula of chlordiazepoxide hydrochloride is as follows:

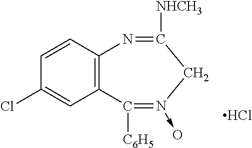

Clidinium bromide is a synthetic anticholinergic agent, having an antispasmodic and antisecretory effect on the gastrointestinal tract. Clidinium bromide is a BCS class II drug and is chemically described as Methyl 5-(propylthio)-2-benzimidazolecarbamate. The structural formula of clidinium bromide is as follows:

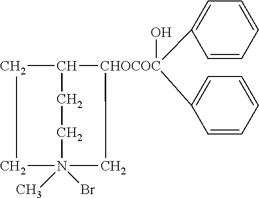

Chlordiazepoxide hydrochloride and clidinium bromide combination is approved as a capsule dosage form under the brand name Librax® in the US and is available in 5 mg and 2.5 mg per unit dose, respectively.

Each Librax® capsule, for oral administration, contains inactive ingredients like corn starch, lactose monohydrate, talc, methylparaben, propylparaben, potassium sorbate, titanium dioxide, and gelatin.

U.S. Pat. No. 2,893,992 assigned to Hoffmann-La Roche discloses chlordiazepoxide or its salts and the process for its preparation.

U.S. Pat. No. 2,648,667 assigned to Hoffmann-La Roche discloses clidinium or its salts and the process for its preparation.

U.S. Pat. No. 3,122,474 assigned to Hoffmann-La Roche discloses a therapeutic composition comprising chlordiazepoxide and clidinium halide. The said patent further discloses that clidinium halide potentiates the depressant effect of chlordiazepoxide, however, no technical data related to stability or incompatibility of both drugs disclosed in this patent publication.

The oral pharmaceutical compositions of chlordiazepoxide hydrochloride and clidinium bromide combination essentially must have desirable chemical and physical properties, purity, dissolution, stability complying with demanding requirements and regulations of health and medicine regulatory agencies across the world.

The development of a combination dosage form of chlordiazepoxide hydrochloride and clidinium bromide is challenging due to multiple factors such as poor content uniformity due to low dose content of chlordiazepoxide hydrochloride (5 mg) and clidinium bromide (2.5 mg); both drugs are prone to degradation in presence of moisture and must be protected from light. Moreover, various undesirable impurities generated during the development of these dosage forms such as:
1. Benzilic acid impurity,
2. Chlordiazepoxide related compound A,
3. 2-Amino-5-chlorobenzophenone,
4. Unknown Impurities of chlordiazepoxide HCl,
5. Unknown Impurities of clidinium Br,
6. Other unknown Impurities,
7. Clidinium bromide related compound A.

The presence of the above-mentioned impurities in the dosage form adversely affects the developed dosage form and accordingly it is desirable to eliminate or keep these impurities within permissible limits. Further, it was observed that under accelerated stability conditions, there is a significant increase in chlordiazepoxide-related compound A impurity when the active pharmaceutical ingredient (API) comes in direct contact with excipients prone to high moisture content. Further, the moisture content of drugs and excipients plays a very important role during the manufacturing of the final product. Moisture content affects the physical, chemical and microbiological properties of pharmaceutical finished dosage forms as well as leads to the degradation of drugs.

In the past, the innovator company of Librax® had to stop distribution and recall marketed formulation due to the presence of undesirable impurities. (https://www.empr.com/uncategorized/recall-for-librax-capsules-and-generic-initiated/).

Therefore, an unmet need exists for compositions of chlordiazepoxide hydrochloride and clidinium bromide having acceptable stability profiles with respect to potency and impurity levels, wherein the composition is substantially free of undesirable impurities. There is also a need for processes for the production of such dosage forms and a test to determine the suitability of excipients and their amounts for the successful development of chlordiazepoxide hydrochloride and clidinium bromide formulations with desired formulation technical attributes.

To overcome the above-mentioned technical problems, inventors focused on identification and mitigation of technical problem by the appropriate selection of excipients and dosage manufacturing conditions and successfully designed pharmaceutical compositions of chlordiazepoxide hydrochloride and clidinium bromide substantially free from undesirable impurities with desirable formulation technical attributes.

Inventors of the present invention discovered that undesirable impurities like chlordiazepoxide related compound A, clidinium bromide related compound A and others can be successfully controlled in the dosage form by: a) using low moisture excipients; and/or b) formulation development under controlled temperature (like not more than 25° C.) and controlled humidity conditions (like relative humidity of less than about 55% and, more particularly, less than about 40%); and/or c) using suitable desiccant in packaging; and/or d) using a specific ratio of drug(s) to selected excipient(s) (like drug(s) to low moisture excipient(s)); and/or e) use of peroxide free excipients.

### SUMMARY OF THE INVENTION

It is an object of the present invention to develop a stable pharmaceutical composition comprising a combination of chlordiazepoxide and clidinium or their pharmaceutically acceptable salts thereof, wherein both the drugs are in single dosage form having desired release profile.

Another object of the present invention is to develop a stable pharmaceutical composition comprising a combination of chlordiazepoxide and clidinium or their pharmaceutically acceptable salts thereof, one or more low moisture excipients, and optionally one or more other pharmaceutically acceptable excipients, wherein the composition is in the form of capsule or sachet dispensed into suitable packaging material.

Yet another object of the invention is to develop a stable pharmaceutical composition of chlordiazepoxide and clidinium combination, which exhibited desirable technical attributes like stability, dissolution, assay, content uniformity, and related substances within pharmaceutically acceptable limits.

### DESCRIPTION OF THE INVENTION

The present invention can be more readily understood by reading the following detailed description of the invention and study of the included examples.

As used herein, the term “composition”, as in pharmaceutical composition, is intended to encompass a drug product comprising an active or its pharmaceutically acceptable salt or derivative thereof, and the other inert ingredient(s) (pharmaceutically acceptable excipients). Such pharmaceutical compositions are synonymous with “formulation” and “dosage form”. The pharmaceutical composition of the invention includes, but is not limited to, capsules, tablets, powder for suspension, powder for solution, ready to use suspension, and ready to use solution. Preferably, the pharmaceutical composition refers to capsules or tablets or sachets.

“Chlordiazepoxide” as used herein refers to the free acid form, its salts, esters, solvates, polymorphs, enantiomers, derivatives, metabolites, or mixtures thereof. Preferably, the salt of chlordiazepoxide is hydrochloride.

“Clidinium” as used herein refers to the free acid form, its salts, esters, solvates, polymorphs, enantiomers, derivatives, or mixtures thereof. Preferably, the salt of clidinium is bromide.

The term “excipient” or “pharmaceutically acceptable excipients” means a pharmacologically inactive component such as binders, diluents, disintegrants, antioxidants, lubricants, glidants, surfactants, wetting agents, solubilizers, stabilizers, anticaking agents, coloring agent, and a carrier or the like. The excipients that are useful in preparing a pharmaceutical composition are generally safe, non-toxic, and are acceptable for veterinary as well as human pharmaceutical use. Reference to an excipient includes both one and more than one such excipient. Co-processed excipients are also covered under the ambit of the present invention. Further, the excipients may be in the form of powders or in the form of a dispersion. Combination of excipients performing the same function may be used to achieve desired formulation characteristics.

The term “low moisture excipient” as used herein refers to an excipient having a moisture content of less than 2%.

The term “controlled atmospheric conditions” refers to a temperature of less than about 25° C., and a relative humidity of less than about 55%, more preferably less than about 40%.

As used herein, the term “about” means ±approximately 20% of the indicated value, such that “about 10 percent” indicates approximately 08 to 12 percent. The term “w/w” as used herein refers to the total weight of the composition.

The term “stable” refers to the compositions of the present invention, wherein substantially no analogues or degradation product of any active pharmaceutical excipient (API) is generated during storage of the dosage form for at least 1 month, preferably for at least 3 months, more preferably for at least 6 months. The stability of the solid oral composition may be evaluated at “long term” conditions 25° C./60% relative humidity, at intermediate condition 30° C./65% relative humidity, at “accelerated conditions” 40° C./75% relative humidity in the final container or pack either measured as the assay or drop in dissolution. Stability testing may be conducted according to the current guidelines by ICH and USFDA.

The term “symmetrical and sequential mixing” as used herein refers to a uniform mixing process wherein the low dose active ingredient(s) and one or more low moisture excipients and/or other pharmaceutically acceptable excipients are mixed in a precise, progressive, or geometric and optimized proportion, particularly in a specific ratio and order.

The term “homogeneous powder mixture” as used herein refers to a uniform and even dispersion of active ingredients with one or more inert excipients like low moisture grade excipients, binders, diluents, surfactants, disintegrants, lubricants, and glidants so that any portion of a final blend sample will exhibit the same concentration of active ingredients i.e., the drug is uniformly distributed in the whole blend.

The term “blend” as used herein refers to a mixture of drug and one or more excipients prepared using progressive or geometric or sequential and in optimized proportion, particularly in a specific ratio and order as described herein.

A first aspect of the present invention relates to a stable pharmaceutical composition comprising:
a) about 0.01% to about 10% by weight of chlordiazepoxide hydrochloride;
b) about 0.01% to about 10% by weight of clidinium bromide;
c) about 5% to about 90% by weight of one or more low moisture excipients;
d) about 0.5% to about 85% by weight of one or more other pharmaceutically acceptable excipients and
wherein the ratio of chlordiazepoxide hydrochloride, clidinium bromide, and the low moisture excipient ranges from about 1:0.5:3 to about 1:0.5:35.

A second aspect of the present invention relates to a stable pharmaceutical composition, wherein the low moisture excipients are free of peroxide.

A third aspect of the present invention relates to a stable pharmaceutical composition comprising:
a) about 0.01% to about 10% by weight of chlordiazepoxide hydrochloride;
b) about 0.01% to about 10% by weight of clidinium bromide;
c) about 5% to about 90% by weight of one or more low moisture excipients selected from corn starch, microcrystalline cellulose, lactose anhydrous, or mixture thereof;
d) about 0.5% to about 85% by weight of one or more other pharmaceutically acceptable excipients selected from lactose, magnesium carbonate, magnesium oxide, magnesium stearate, talc and
wherein the ratio of chlordiazepoxide hydrochloride, clidinium bromide, and the low moisture excipient ranges from about 1:0.5:3 to about 1:0.5:35 and the composition is prepared under a relative humidity of less than 55%, wherein the moisture content of low moisture excipient is less than 2% and the D₉₀ value of chlordiazepoxide hydrochloride and clidinium bromide is less than about 100 μm and the D₉₀ value of low moisture excipient is less than about 250 μm.

A fourth aspect of the present invention relates to a stable pharmaceutical composition comprising a combination of chlordiazepoxide and clidinium or their pharmaceutically acceptable salts thereof, wherein the low moisture excipient is selected from lactose anhydrous, microcrystalline cellulose, corn starch, silicified microcrystalline cellulose, sodium carboxymethyl cellulose, mannitol or mixtures thereof.

A fifth aspect of the present invention relates to a stable pharmaceutical composition comprising a combination of chlordiazepoxide and clidinium or their pharmaceutically acceptable salts thereof, and at least one or more pharmaceutically acceptable excipients selected from calcium carbonate, calcium phosphate, dibasic anhydrous calcium phosphate, calcium sulphate, lactose monohydrate, magnesium carbonate, magnesium oxide, maltodextrin, maltose, xylitol, sorbitol, pregelatinized starch, magnesium stearate, talc, sucrose or mixtures thereof.

A six aspect of the present invention relates to a stable pharmaceutical composition comprising:
a) about 0.01% to about 10% by weight of chlordiazepoxide or its pharmaceutically acceptable salts thereof;
b) about 0.01% to about 10% by weight of clidinium or its pharmaceutically acceptable salts thereof;
c) about 5% to about 90% by weight of one or more low moisture excipients selected from corn starch, microcrystalline cellulose, lactose anhydrous, or a mixture thereof; and
d) about 0.5% to about 85% by weight of one or more other pharmaceutically acceptable excipients selected from lactose monohydrate, magnesium carbonate, magnesium oxide, magnesium stearate, and talc,
wherein the ratio of chlordiazepoxide or its salts; clidinium or its salts; the low moisture excipient ranges from about 1:0.5:3 to about 1:0.5:35 and the composition is prepared under a relative humidity of less than 55%, wherein the moisture content of low moisture excipient is less than 2% and the D₉₀ value of chlordiazepoxide and clidinium is less than about 100 μm and the D₉₀ value of low moisture grade excipient is less than about 250 μm.

A seventh aspect of the present invention relates to a stable pharmaceutical composition comprising a combination of chlordiazepoxide and clidinium or their pharmaceutically acceptable salts thereof, wherein the pharmaceutical composition is stable for at least 6 months at 40° C. and 75% relative humidity, and wherein the composition has a total impurity content of less than 1.5%.

An eighth aspect of the present invention relates to a stable pharmaceutical composition comprising a combination of chlordiazepoxide and clidinium or their pharmaceutically acceptable salts thereof, wherein the process of preparing the pharmaceutical composition is selected from dry blending, direct compression, symmetrical and sequential mixing, dry granulation, hot melt extrusion, extrusion spheronization or spray drying. In a preferred embodiment, the pharmaceutical composition is prepared by dry blending.

A ninth aspect of the present invention relates to a stable pharmaceutical composition comprising a combination of chlordiazepoxide and clidinium or their pharmaceutically acceptable salts thereof, wherein the composition is in the form of capsules, tablets, powder for oral suspension, powder for oral solution, ready to use suspension or ready to use solution, sprinkles, granules, and multilayer tablets.

A tenth aspect of the present invention relates to a stable pharmaceutical composition comprising a combination of chlordiazepoxide and clidinium or their pharmaceutically acceptable salts thereof, wherein the moisture content of the pharmaceutical composition is less than 6%, determined using the Karl-Fischer method as disclosed in USP <921> direct titration (Method 1a).

An eleventh aspect of the present invention relates to a process for preparing a pharmaceutical composition comprising:
a) mixing of about 0.01% to about 10% by weight of chlordiazepoxide hydrochloride and about 0.01% to about 10% by weight of clidinium bromide with about 10% to about 30% by weight of one or more low moisture excipients;
b) co-mixing the mixture of step a) with about 20% to about 35% by weight of one or more other pharmaceutically acceptable excipients;
c) sifting the mixture of step b) through a suitable size mesh;
d) adding optionally one or more other pharmaceutically acceptable excipients to the homogeneous powder mixture of step c), and
e) filling the homogeneous powder mixture of step d) into the suitable size of capsule shell or sachet;
wherein the composition is prepared under a relative humidity of less than 40% and temperature not more than 25° C.

A twelfth aspect of the present invention relates to a stable pharmaceutical composition comprising a combination of chlordiazepoxide and clidinium or their pharmaceutically acceptable salts thereof, wherein the said composition is packed in a packaging material selected from the group consisting of a blister, bottle, container, and wherein the packaging material contains one or more desiccants. In certain non-limiting embodiments, the desiccant is silica gel.

A thirteenth aspect of the present invention relates to a stable pharmaceutical composition comprising a combination of chlordiazepoxide and clidinium or their pharmaceutically acceptable salts thereof, wherein the composition is used to control emotional and somatic factors in gastrointestinal disorders and as adjunctive therapy in the treatment of peptic ulcer and in the treatment of the irritable bowel syndrome (irritable colon, spastic colon, mucous colitis) and acute enterocolitis.

A fourteenth aspect of the present invention relates to a stable pharmaceutical composition comprising a combination of chlordiazepoxide and clidinium or their pharmaceutically acceptable salts thereof, wherein the composition is a capsule or sachet.

A fifteenth aspect of the present invention relates to a stable pharmaceutical composition comprising a combination of chlordiazepoxide and clidinium or their pharmaceutically acceptable salts thereof, wherein the particle size of the drugs is having D₉₀ value less than about 100 μm and the low moisture excipient is having D₉₀ value less than about 250 μm as determined by Malvern Particle Size Analyzer.

A sixteenth aspect of the present invention relates to a stable pharmaceutical composition comprising a combination of chlordiazepoxide and clidinium or their pharmaceutically acceptable salts thereof, wherein the dose of chlordiazepoxide or its pharmaceutically acceptable salts thereof is about 1 mg to about 10 mg, preferably about 5 mg and clidinium or its pharmaceutically acceptable salts thereof is from about 1 mg to 10 mg, preferably about 2.5 mg.

Another aspect of the present invention relates to a stable pharmaceutical composition comprising a combination of chlordiazepoxide and clidinium or their pharmaceutically acceptable salts thereof, exhibited drug release comparable to the equivalent dose of marketed dosage form, wherein 95% of the drug is released in water (900 mL) within 30 minutes using a USP I apparatus at 100 rpm.

Another aspect of the present invention relates to a stable pharmaceutical composition, wherein excipients can optionally be pre-treated to remove extra moisture. Another aspect of the present invention relates to a stable pharmaceutical composition, wherein excipients are pre-treated to remove extra moisture using techniques like heat treatment of excipients in an oven or using desiccator, desiccant, or adsorbents.

Another aspect of the present invention relates to a stable pharmaceutical composition comprising a combination of chlordiazepoxide and clidinium or their pharmaceutically acceptable salts thereof, wherein the drug load is about 0.01% to about 7% by weight, preferably about 1% to about 5% by weight, and wherein, the composition exhibits desirable technical attributes like assay, content uniformity, dissolution, stability, and related substances.

Yet another aspect of the present invention relates to a stable pharmaceutical composition comprising a combination of chlordiazepoxide and clidinium or their pharmaceutically acceptable salts thereof, having a bulk density in the range from about 0.525 g/cc to about 0.750 g/cc and tapped density in the range from about 0.825 g/cc to about 0.99 g/cc.

Another aspect of the present invention relates to a stable pharmaceutical composition comprising a combination of chlordiazepoxide and clidinium or their pharmaceutically acceptable salts thereof, which is stable at 40° C. and 75%±5% relative humidity at least for a period of about 3 months.

Another aspect of the present invention relates to a stable pharmaceutical composition, wherein the composition is prepared at a temperature, not more than 25° C. and under a relative humidity of less than 55% in presence of sodium vapor lamp, preferably the composition is prepared at a temperature, not more than 25° C. and under a relative humidity of less than 40% in presence of sodium vapor lamp. In a certain non-limiting embodiment, all the portions are blended together.

Yet another aspect of the present invention relates to a stable pharmaceutical composition comprising a combination of chlordiazepoxide and clidinium or their pharmaceutically acceptable salts thereof, wherein the one or more low moisture grade excipients are selected from the group consisting of lactose such as lactose anhydrous, Lactopress®, microcrystalline cellulose, corn starch, Unipure® F, Unipure® FL, silicified microcrystalline cellulose, sodium carboxymethyl cellulose, mannitol, xylitol, or mixtures thereof, preferably, lactose anhydrous, corn starch, Lactopress®, Unipure® F, Unipure® FL, silicified microcrystalline cellulose and mannitol or mixtures, thereof.

Another aspect of the present invention relates to a stable pharmaceutical composition comprising a combination of chlordiazepoxide and clidinium or their pharmaceutically acceptable salts thereof, wherein the active ingredients and one or more low moisture grade excipients have a ratio ranging from about 1:10 to about 1:50 by weight; preferably from about 1:15 to about 1:40 by weight.

In another aspect of the invention, dry blending can be performed by using V-blender, double cone mixer, paddle blender, key blender, powder mixer, mass mixer, or by any other appropriate method known in the art.

In another embodiment of the present invention, the capsule shell can be gelatin based or HPMC (hydroxypropyl methylcellulose or hypromellose) based or PVA (Polyvinl acetate) based or cellulose ether film based or starch based.

Various low moisture grade excipients include, but are not limited to microcrystalline cellulose, silicified microcrystalline cellulose, calcium carbonate, calcium phosphate dibasic anhydrous, calcium phosphate dibasic dihydrate, calcium sulphate, magnesium carbonate, magnesium oxide, maltodextrin, maltose, mannitol, xylitol, sorbitol, starch, pregelatinized starch, sucrose, lactose anhydrous, Lactopress®, corn starch, Unipure F, Unipure FL, sodium carboxymethyl cellulose, mannitol, xylitol, or mixtures thereof, preferably lactose anhydrous, corn starch, Lactopress®, Unipure® F, Unipure® FL or mixtures thereof. The low moisture grade excipients may be present in an amount of about 0.001% to about 98% w/w.

Binders impart cohesiveness to formulation, various useful binders include, but are not limited to celluloses derivatives such as hydroxypropylcellulose (HPC), hydroxypropylmethylcellulose (HPMC), methyl cellulose, ethyl cellulose, hydroxyethyl cellulose, carboxymethylcellulose, sodium carboxymethylcellulose, a mixture of microcrystalline cellulose and carboxymethylcellulose (Avicel® RC591), acacia, alginic acid, carbomer, dextrin, gelatin, glucose, guar gum, maltose, povidone, vinylpyrolidone vinyl acetate copolymer (PVP/VA) polymers, copovidone, starch, polyvinyl alcohol or polyethylene oxide, or mixtures thereof. The binder may be present in an amount of about 0.01% to about 40% w/w, preferably about 0.1% to about 35% w/w.

Diluents or fillers or carriers are substances that usually provide bulk to the composition. Various useful fillers or diluents include, but are not limited to microcrystalline cellulose, silicified microcrystalline cellulose, calcium carbonate, calcium phosphate, dibasic anhydrous, calcium phosphate dibasic dihydrate, calcium phosphate tribasic, calcium sulphate, lactose, lactose monohydrate, magnesium carbonate, magnesium oxide, maltodextrin, maltose, mannitol, xylitol, sorbitol, starch, pregelatinized starch, sucrose or mixtures thereof. The diluent may be present in an amount of about 5% to about 95% w/w, preferably about 20% to about 85% w/w.

Disintegrants according to the present invention are selected from the group comprising crospovidone, modified starches, croscarmellose sodium, sodium starch glycolate, low substituted hydroxypropyl cellulose, microcrystalline cellulose, corn starch, pregelatinized starch, and carboxymethylcellulose calcium. These disintegrants are also known as super-disintegrants. The disintegrant may be present in an amount of about 1% w/w to about 40% w/w, preferably from about 1% w/w to about 30% w/w.

Surfactants contemplated in the present invention include but are not limited to anionic surfactants, amphoteric surfactants, non-ionic surfactants, and macromolecular surfactants. Suitable examples of surfactants include but are not limited to sodium lauryl sulphate, lecithin, stearyl alcohol, cetyl stearyl alcohol, polyoxyethylene sorbitan fatty acid esters such as polysorbate 80, polysorbate 20, Poloxamer 188, polyoxyethylene fatty acid glycerides such as macrogol 1000 glycerol monostearate, polyoxyethylene fatty acid esters such as polyoxyl 40 stearate, polyoxyethylene fatty alcohol ethers such as polyoxyl 10 oleyl ether, and glycerol fatty acid esters such as glycerol monostearate. The surfactant may be present in an amount of about 0.5% w/w to about 10% w/w, preferably from about 1% w/w to about 5% w/w and more preferably from about 0.5% w/w to about 2% w/w.

Glidants improve flowability and accuracy of dosing. Glidants used in the composition include, but are not limited to, tribasic calcium phosphate, calcium silicate, cellulose powdered, colloidal silicon dioxide, magnesium silicate, magnesium trisilicate, starch, and talc or mixtures thereof. The amount of glidant ranges from about 0.1% to about 5% w/w of the total composition.

Various useful preservatives according to the present invention include, but are not limited to, parabens such as methylparaben, propylparaben, butyl paraben or their salts, benzoic acid or its salts, sodium benzoate, potassium benzoate, calcium benzoate, methyl hydroxybenzoate, ethyl para-hydroxybenzoate, sodium ethyl para-hydroxybenzoate, sodium metabisulphite, chlorhexidine, diazolidinyl urea, sodium citrate, citric acid butylated hydroxyl toluene (BHT), butylated hydroxyl anisole (BHA), tocopherol, and the like. In particular, the preservative is selected from benzoic acid or its salts, butylated hydroxyl toluene (BHT), butylated hydroxyl anisole (BHA), and parabens. The preservative may be present in an amount of about 0.5% w/w to about 10% w/w, preferably from about 0.5% w/w to about 2% w/w.

Suitable coloring agents according to the present invention are selected from the group comprising FD&C (Federal Food, Drug and Cosmetic Act) approved coloring agents, natural coloring agents, natural juice concentrates, pigments such as iron oxide, titanium dioxide, and zinc oxide, and combinations thereof. The coloring agent may be present in an amount of about 0.1% to about 2% w/w, preferably to about 0.3% w/w to about 1% w/w.

The formulations according to the present invention may be filled in capsules and may be coated or uncoated. For coating, additional excipients such as film-forming polymers or membrane forming agents both water-soluble polymers and water-insoluble polymers or acrylate, plasticizers, anti-adherents, and opacifiers are used.

Various water-soluble polymers are used to form a barrier/seal or film over the core. Examples include but are not limited to cellulose derivatives such as soluble alkyl- or hydroalkylcellulose derivatives such as methylcellulose, hydroxymethyl cellulose, hydroxyethyl cellulose, hydroxypropyl cellulose, hydroxymethylethyl cellulose, hydroxypropyl methylcellulose, vinylpyrrolidone vinyl acetate copolymer (PVP/VA) polymers, sodium carboxymethyl cellulose, etc., polyvinyl alcohol, polyvinyl acetate, polyvinylpyrrolidone, chitosan and derivatives thereof, shellac and derivatives thereof, waxes and fat substances. The water-soluble polymers may be present in an amount of about 1% w/w to about 80% w/w, preferably from about 5% w/w to about 70% w/w and more preferably from about 10% w/w to about 60% w/w.

Examples of a coating method include, for example, using a coating pan, or a fluidized bed.

In another embodiment, the present invention includes particle size of free drug particulate form of chlordiazepoxide and clidinium or their pharmaceutically acceptable salts thereof, wherein particle diameter at 90% cumulative volume (d₉₀) is less than about 100 μm and the one or more low moisture grade excipients is having D₉₀ value less than about 250 μm. Particle diameter at X % cumulative particle size reduction can be performed by techniques including but not limited to fluid energy milling, ball milling, colloid milling, roller milling, Quadro co-mill, hammer milling, and the like. Particle size and particle size distribution can be measured by techniques such as Laser light scattering (e.g. Malvern Light Scattering) using Malvern particle size analyzer, Coulter counter, microscopy, and the like.

Relative Humidity or air humidity as well as air temperature, dew point temperature, and wet-bulb temperature are measured using a digital hygrometer and/or digital thermometer, and/or digital relative humidity meters. Air conditioning and handling equipment based on refrigeration cycles—heating ventilation and air conditioning (HVAC) systems, portable dehumidifiers are used to build the working environments to maintain levels of relative humidity between 40 to 60%, with temperatures around 21-25° C.

The pharmaceutical oral dosage form prepared according to the present invention can be subjected to in vitro dissolution evaluation according to Test 711 “Dissolution” in the United States Pharmacopoeia 37, United States Pharmacopoeial Convention, Inc., Rockville, Md., 2014 (“USP”) to determine the rate at which the active substance is released from the dosage form, and the content of the active substance can be determined in solution by high performance liquid chromatography. When comparing the test and reference products, dissolution profiles should be compared using a similarity factor (f₂). The similarity factor is a logarithmic reciprocal square root transformation of the sum of squared error and is a measurement of the similarity in the percent (%) of dissolution between the two curves.

f₂=50·log{[1+(1/n)Σₜ₌₁ⁿ(Rₜ−Tₜ)²]^{-0.5}·100}

Two dissolution profiles are considered similar when the f2 value is equal to or greater than 50.

In another embodiment, the stable pharmaceutical composition of the present invention exhibited drug release whereby more than 95% of the drug is released in 30 minutes in 900 ml of purified water, sampling at 5, 10, 15, 20, and 30 minutes (as per US Pharmacopoeia mentioned in Office of Generic Drugs dissolution database) using a USP I apparatus (basket) at a temperature of 37±0.5° C. and a rotation speed of 100 revolutions per minute. The resultant liquid has been analyzed in the UV spectrum at about 212 nm wavelength.

Having described the invention with reference to certain preferred embodiments, other embodiments will become apparent to one skilled in the art from consideration of the specification. The present invention is further defined by reference to the following examples describing in detail methods for the preparation and testing of the said pharmaceutical composition. It will be apparent to those skilled in the art that many modifications, both to materials and methods, may be practiced without departing from the scope of the invention. Following examples are set out to illustrate the invention and do not limit the scope of the present invention.

### EXAMPLES

The following non-limiting examples are intended to further illustrate certain preferred embodiments of the invention. They are, however not intended to be limiting to the scope of the present invention. The present inventors prepared a test batch using a wet granulation process without any controlled conditions and surprisingly observed a significantly higher amount of undesirable Chlordiazepoxide related compound A, up to the level of 12.54%.

To understand the scientific rationale of this significant impurity increase, the present inventors performed forced degradation studies as given in table 1:

TABLE 1
Forced degradation studies:

| | | Chlordiazepoxide |
|---|---|---|
| Mode of | | Related Compound- |
| Degradation | Condition | A (% w/w) |
| Control | Room | 0.138 |
| | Temperature | |
| | (RT) | |
| Peroxide | 2 ml/80° C./ | 4.741 |
| Degradation 30% | 40 Min | |
| w/v | | |
| Thermal | 105° C./168 Hrs | 2.125 |
| degradation | | |
| Humidity | 95% RH/168 Hrs | 0.538 |
| Degradation | | |

It was observed that undesirable Chlordiazepoxide related compound A was attributed due to the presence of high moisture, or thermal stress conditions, or under Peroxide exposure.

Accordingly, the present inventors successfully controlled these conditions like a) use of low moisture grade excipient; b) use of peroxide free excipients; c) under controlled temperature and humidity conditions.

Pharmaceutical composition of chlordiazepoxide and clidinium or their pharmaceutically acceptable salts thereof may be prepared by using quantitative formula as given in table 2:

TABLE 2
Batch with low moisture content excipients
(Different grades of Corn starch)

| Sr. | | Example 1 | Example 2 |
|---|---|---|---|
| No | Ingredients | % w/w | |
| 1 | Chlordiazepoxide hydrochloride | 2.5 | 2.50 |
| 2 | Clidinium bromide | 1.25 | 1.25 |
| 3 | Lactose | 71.8 | 71.8 |
| 4 | Corn-starch (unipure-F) | 23.94 | NA |
| | Moisture content - Max. 14% | | |
| | Corn-starch (unipure-FL) | NA | 23.94 |
| | Moisture content - Max. 2% | | |
| 5 | Talc | 0.5 | 0.5 |
| 6 | Size ‘3’ hard gelatin capsule | 01 No's | 01 No's |

Manufacturing Process of Example 1 & Example 2:
1. All the ingredients were weighed accurately.
2. Lactose and corn starch were sifted separately through a suitable size mesh.
3. Chlordiazepoxide hydrochloride, clidinium bromide, and corn starch were co-sifted through a suitable size mesh.
4. Material of step 3 and lactose monohydrate were blended in a blender.
5. Talc was passed through a suitable size mesh.
6. Dried granules of step 4 were sifted along with Talc.
7. The blend of step 6 was filled in to the suitable size hard gelatin capsules or in sachets.

TABLE 3
Stability data of batches with different grades of Corn Starch

| Elements | Example 1 | | Example 2 | |
|---|---|---|---|---|
| Corn starch grade | Unipure-F | | Unipure-FL | |
| | Moisture content - | | Moisture content - | |
| | Max. 14% | | Max. 2% | |
| Condition | Initial | 40° C./75% | Initial | 40° C./75% |
| | | Relative | | Relative |
| | | humidity | | humidity |
| | | (3M) | | (3M) |

Related substance analyzed by High-performance liquid
chromatography (HPLC) (w/w)

| | | | | |
|---|---|---|---|---|
| Chlordiazepoxide | 0.230 | 2.20 | 0.88 | 0.771 |
| related | | | | |
| compound A | | | | |
| 2-Amino 5- | ND | ND | ND | ND |
| chlorobenzophenone | | | | |
| Clidinium related | ND | ND | ND | ND |
| compound A | | | | |
| Highest unknown | ND | ND | ND | ND |
| impurity | | | | |
| Total impurities | 0.230 | 2.20 | 0.88 | 0.771 |

From above table 3, a significant increase in chlordiazepoxide related compound A was observed in example 1 having corn starch with higher moisture content (Unipure F—moisture content 14%) as compared with the formulation of example 2, having corn starch with low moisture content (Unipure FL—moisture content less than 2%).

TABLE 4
Test examples with low moisture grade excipients:

| | Example 3 | Example 4 |
|---|---|---|
| Ingredients | % w/w | |
| Chlordiazepoxide HCl | 2.5 | 2.5 |
| Clidinium bromide | 1.25 | 1.25 |
| Lactose monohydrate | 82.78 | 70.5 |
| Corn Starch with 2% moisture | 11.97 | 23.94 |
| (Unipure - FL) | | |
| Talc | 1.50 | 1.50 |
| Size ‘n3’ Hard gelatin capsule | 01 No's | 01 No's |

TABLE 5
Impact of the content of the low moisture grade excipient(s) on comparative stability
studies:

| | Librax ® | | Example 3 | | Example 4 | |
|---|---|---|---|---|---|---|
| | | 40° C./75% | | 40° C./75% | | 40° C./75% |
| | | Relative | | Relative | | Relative |
| Example | | Humidity | | Humidity | | Humidity |
| Condition | Initial | (6M) | Initial | (6M) | Initial | (6M) |

Related substance by HPLC (w/w)

| | | | | | | |
|---|---|---|---|---|---|---|
| Chlordiazepoxide | 1.165 | 4.690 | 0.163 | 2.785 | 0.126 | 2.023 |
| related compound A | | | | | | |
| 2-Amino 5- | ND | ND | ND | ND | ND | ND |
| chlorobenzophenone | | | | | | |
| Clidinium related | ND | ND | ND | ND | ND | ND |
| compound A | | | | | | |
| Highest unknown | 0.110 | 0.465 | ND | ND | ND | ND |
| impurity | | | | | | |
| Total impurities | 1.275 | 5.155 | 0.163 | 2.785 | 0.126 | 2.023 |

ND = Not Detected.

Under controlled humidity conditions and use of low moisture grade excipient in formulation process facilitated a significant reduction in undesirable impurities content in comparison to marketed Librax® formulation. Further, inventors surprisingly found that by using a higher amount of low moisture grade excipient (starch Unipure—FL), a further significant decrease in Chlordiazepoxide related compound A impurity was observed as compared to the marketed Librax® formulation and test example 3.

TABLE 6
Capsule Formulation by using dry blending

| Ingredients | | Example 5 | Example 6 | Example 7 | Example 8 |
|---|---|---|---|---|---|
| Name | Role | Quantity % w/w | | | |
| Chlordiazepoxide | Active | 2.5 | 2.5 | 2.5 | 2.5 |
| Hydrochloride | | | | | |
| Clidinium | Active | 1.25 | 1.25 | 1.25 | 1.25 |
| Bromide | | | | | |
| Lactose | Diluent | — | 83.78 | — | 83.78 |
| monohydrate | | | | | |
| Lactose | Diluent | 82.78 | — | 83.78 | — |
| anhydrous | | | | | |
| Corn Starch | Low | 11.97 | 11.97 | 11.97 | 11.97 |
| | moisture | | | | |
| | excipient | | | | |
| Talc | Glidant | 3.0 | 1.0 | 1.0 | 1.0 |
| | Conditions during manufacturing | | | | |
| Humidity | | NMT* 55% | NMT* 40% | NMT* 40% | NMT* 40% |
| during process | | | | | |

*Not more than

The Dry Blending Process of Example 5-8 Includes Following Steps:
1. Lactose sifted through a suitable size mesh.
2. Chlordiazepoxide hydrochloride, clidinium bromide, and corn starch were co-sifted through a suitable size mesh.
3. Material of step 1 was co-sifted with step 2 through a suitable size mesh.
4. Material of step 3 was co-sifted and loaded into the blender.
5. Lactose and talc were co-sifted through suitable size mesh and loaded into the blender containing a blend of step 4 and blended together.
6. The blend of step 5 was filled in to the suitable size hard gelatin capsules or in sachets.

To study the effect of moisture on the stability of formulations, one part of the blends of examples 7 and 8 were exposed to 60% relative humidity for 8 hours (at 25° C.) during capsule filling and significant degradation was observed in the formulation exposed to humidity. The LOD at 105° C. of the formulations i.e., example 7 and example 8 were less than 1.9% w/w, preferably less than 1.0% w/w, respectively. The bulk density of chlordiazepoxide hydrochloride, clidinium bromide, formulation of example 7 and example 8 are less than 0.70 g/cc.

The above-mentioned examples 7 and 8 and the exposed samples (initial and 40° C./75% relative humidity for 1 month samples) exhibited the in-vitro dissolution profile, assay, and related substances as shown in Table 7 and Table 8:

TABLE 7
Dissolution data and Assay data at initial and 40° C./75% relative humidity
(1 month) stability for test and reference products:

| | | | | Example 7 (a) | |
|---|---|---|---|---|---|
| | | | | (exposed to | |
| | | Example 7 | | humidity) | |
| | | | 40° C./75% | | 40° C./75% |
| | Reference | | relative | | relative |
| Example | (LIBRAX ®) | | humidity | | humidity |
| Condition | Initial | Initial | (1 Month) | Initial | (1 Month) |
| Assay (%) | 99.28 | 101.27 | 97.07 | 97.67 | 96.69 |
| Chlordiazepoxide | | | | | |
| HCl | | | | | |
| Assay (%) of | 101.93 | 102.12 | 97.35 | 98.22 | 96.96 |
| Clidinium Br | | | | | |
| Time | Cumulative % drug release of chlordiazepoxide | | | | |
| (Minutes) | hydrochloride in Purified Water, 900 ml, USP-I | | | | |
| 10 | 88 | 99 | 94 | 95 | 94 |
| 15 | 99 | 102 | 100 | 99 | 100 |
| 20 | 102 | 102 | 100 | 99 | 100 |
| 30 | 102 | 103 | 100 | 98 | 100 |
| Time | Cumulative % drug release of clidinium bromide in Purified | | | | |
| (Minutes) | Water, 900 ml, USP-I | | | | |
| 10 | 95 | 93 | 95 | 96 | 95 |
| 15 | 101 | 97 | 101 | 100 | 99 |
| 20 | 103 | 98 | 98 | 101 | 99 |
| 30 | 102 | 96 | 100 | 101 | 99 |

Related substance (By HPLC)

| | | | | | |
|---|---|---|---|---|---|
| Impurity A | 1.165 | 0.145 | 1.061 | 0.159 | 1.150 |
| 2-Amino-5- | ND | ND | ND | ND | ND |
| chlorobenzophenone | | | | | |
| Highest unknown | 0.110 | ND | ND | ND | ND |
| impurity | | | | | |
| Total Impurity | 1.275 | 0.145 | 1.061 | 0.159 | 1.150 |

TABLE 8
Dissolution data and Assay data at initial and 40° C./75% relative humidity (1 month)
stability for test and reference products:

| | | | | Example 8 (a) | |
|---|---|---|---|---|---|
| | | Example 8 | | (exposed to humidity) | |
| | | | 40° C./75% | | 40° C./75% |
| | Reference | | relative | | relative |
| Example | (LIBRAX ®) | | humidity | | humidity |
| Condition | Initial | Initial | (1 Month) | Initial | (1 Month) |
| Assay (%) | 99.28 | 98.81 | 97.01 | 99.31 | 98.11 |
| Chlordiazepoxide | | | | | |
| hydrochloride | | | | | |
| Assay (%) of | 101.93 | 99.1 | 96.68 | 100.35 | 98.25 |
| Clidinium | | | | | |
| bromide | | | | | |
| Time | Cumulative % drug release of chlordiazepoxide HCl in Purified | | | | |
| (Minutes) | Water, 900 ml, USP-I | | | | |
| 10 | 88 | 102 | 96 | 92 | 96 |
| 15 | 99 | 104 | 99 | 95 | 97 |
| 20 | 102 | 105 | 100 | 96 | 97 |
| 30 | 102 | 105 | 100 | 96 | 97 |
| Time | Cumulative % drug release of clidinium Br in Purified Water, | | | | |
| (Minutes) | 900 ml, USP-I | | | | |
| 10 | 95 | 105 | 100 | 97 | 98 |
| 15 | 101 | 108 | 100 | 100 | 100 |
| 20 | 103 | 107 | 101 | 98 | 99 |
| 30 | 102 | 106 | 101 | 99 | 99 |

Related substance (By HPLC)

| | | | | | |
|---|---|---|---|---|---|
| Impurity A | 1.165 | 0.139 | 1.071 | 0.143 | 1.194 |
| 2-Amino-5- | ND | ND | ND | ND | ND |
| chlorobenzophenone | | | | | |
| Highest | 0.110 | ND | ND | ND | ND |
| unknown | | | | | |
| impurity | | | | | |
| Total Impurity | 1.275 | 0.139 | 1.071 | 0.143 | 1.194 |

Accelerated stability testing as per the ICH guidelines was conducted on the capsules prepared in examples 7 and 8 and comparative examples 7 (a) and 8 (a) at temperature/relative humidity of 40° C.±2° C./75%±5% for 1 month. Results in terms of the amount of chlordiazepoxide hydrochloride and clidinium bromide capsule of the period of storage analyzed by validated high performance liquid chromatography.

It was observed that by controlling the humidity during the manufacturing process and filling of capsules, the level of total impurity significanly decreased as compared to the reference product. Inventors of the present invention found that by selecting optimized processes and using low moisture excipients, they have stabilized the active ingredients as well as the finished products and resolved the content uniformity issues.

In view of the above, it is evident that the formulation of the invention provides an in vitro dissolution of chlordiazepoxide hydrochloride and clidinium bromide comparable to Librax® (marketed) capsules.

While this invention has been described in detail with reference to certain preferred embodiments, it should be appreciated that the present invention is not limited to those precise embodiments. Rather, in view of the present disclosure, which describes the current best mode for practicing the invention, many modifications and variations would present themselves to those skilled in the art without departing from the scope and spirit of the invention.

What is claimed:

1. A stable pharmaceutical composition comprising:
a) about 0.01% to about 10% by weight of chlordiazepoxide hydrochloride;
b) about 0.01% to about 10% by weight of clidinium bromide;
c) about 5% to about 90% by weight of one or more low moisture excipients;
d) about 0.5% to about 85% by weight of one or more other pharmaceutically acceptable excipients and
wherein the ratio of chlordiazepoxide hydrochloride, clidinium bromide, and the low moisture excipient ranges from about 1:0.5:3 to about 1:0.5:35.

2. The stable pharmaceutical composition according to claim 1, wherein the low moisture excipients are free of peroxide.

3. The stable pharmaceutical composition according to claim 1, wherein the low moisture excipients are selected from lactose anhydrous, corn starch, silicified microcrystalline cellulose, mannitol, sodium carboxymethyl cellulose, or mixtures thereof.

4. The stable pharmaceutical composition according to claim 1, wherein the particle size of the chlordiazepoxide hydrochloride and clidinium bromide is having D₉₀ value less than about 100 μm.

5. The stable pharmaceutical composition according to claim 1, wherein the particle size of the low moisture excipients is having D₉₀ value less than about 250 μm.

6. The stable pharmaceutical composition according to claim 1, wherein the other pharmaceutically acceptable excipients are selected from calcium carbonate, calcium phosphate, dibasic anhydrous calcium phosphate, calcium sulphate, lactose, magnesium carbonate, magnesium oxide, maltodextrin, maltose, xylitol, sorbitol, pregelatinized starch, magnesium stearate, talc, sucrose or mixtures thereof.

7. A stable pharmaceutical composition comprising:
a) about 0.01% to about 10% by weight of chlordiazepoxide hydrochloride;
b) about 0.01% to about 10% by weight of clidinium bromide;
c) about 5% to about 90% by weight of one or more low moisture excipients selected from corn starch, microcrystalline cellulose, lactose anhydrous, or mixture thereof; and
d) about 0.5% to about 85% by weight of one or more other pharmaceutically acceptable excipients selected from lactose, magnesium carbonate, magnesium oxide, magnesium stearate, and talc
wherein the ratio of chlordiazepoxide hydrochloride, clidinium bromide, and the low moisture excipient ranges from about 1:0.5:3 to about 1:0.5:35 and the composition is prepared under a relative humidity of less than 55%, wherein the moisture content of low moisture excipient is less than 2% and the D₉₀ value of chlordiazepoxide hydrochloride and clidinium bromide is less than about 100 μm and the D₉₀ value of low moisture excipient is less than about 250 μm.

8. The stable pharmaceutical composition according to claim 7, wherein the composition is prepared under a relative humidity of less than 40% and temperature not more than 25° C.

9. The stable pharmaceutical composition according to claim 1, wherein the pharmaceutical composition is stable for at least 6 months at 40° C. and 75% relative humidity, and wherein the composition has a total impurity content of less than 1.5%.

10. The stable pharmaceutical composition according to claim 1, wherein the process of preparing the pharmaceutical composition is selected from dry blending, symmetrical and sequential mixing, dry granulation, or spray drying.

11. The stable pharmaceutical composition according to claim 1, wherein the composition is in the form of capsules, powder for oral suspension, powder for oral solution, ready to use suspension, ready to use solution, sprinkles, and granules.

12. The stable pharmaceutical composition according to claim 1, wherein the moisture content of the composition is less than 6%, as measured using the Karl-Fischer method.

13. A process for preparing a stable pharmaceutical composition comprising:
a) mixing of about 0.01% to about 10% by weight of chlordiazepoxide hydrochloride and about 0.01% to about 10% by weight of clidinium bromide with about 10% to about 30% by weight of one or more low moisture excipients;
b) co-mixing the mixture of step a) with about 20% to about 35% by weight of one or more other pharmaceutically acceptable excipients;
c) sifting the mixture of step b) through a suitable size mesh;
d) adding optionally one or more other pharmaceutically acceptable excipients to the homogeneous powder mixture of step c), and
e) filling the homogeneous powder mixture of step d) into the suitable size of capsule shell or sachet;
wherein the composition is prepared under a relative humidity of less than 40% and temperature not more than 25° C.

14. The stable pharmaceutical composition according to claim 13, wherein the process of preparing the pharmaceutical composition comprises symmetrical and sequential mixing.

15. The stable pharmaceutical composition according to claim 13, wherein the said composition is a capsule or sachet.

16. The stable pharmaceutical composition according to claim 1, wherein the said composition exhibits drug release comparable to the equivalent dose of marketed dosage form wherein 95% drug is released in water (900 mL) within 30 minutes using a USP I apparatus at 100 rpm.

17. The stable pharmaceutical composition according to claim 1, wherein the composition is packed in a packaging material selected from the group consisting of a blister, bottle, and container wherein the packaging material contains one or more desiccants.

18. The stable pharmaceutical composition according to claim 17, wherein desiccant is silica gel.

19. The stable pharmaceutical composition according to claim 1, wherein excipients can optionally be pre-treated to remove extra moisture.

20. The stable pharmaceutical composition according to claim 19, wherein excipients are pre-treated to remove extra moisture using techniques like heat treatment of excipients in an oven or using desiccator, desiccant or adsorbents.
